(19) United States
(12) Patent Application Publication
(10) Pub. No.: US 2002/0082553 A1
(43) Pub. Date: Jun. 27, 2002

(54) Balloon designs for angioplasty

(75) Inventor: Jacky G. Duchamp, Campbell, CA (US)

Correspondence Address: HELLER EHRMAN WHITE & MCAULIFFE LLP, 275 MIDDLEFIELD ROAD, MENLO PARK, CA, 94025-3506, US

(73) Assignee: Advanced Cardiovascular Systems, Inc.

(21) Appl. No.: 09/747,553

(22) Filed: Dec. 22, 2000

(51) Int. Cl. A61M 29/00

(52) U.S. Cl. 604/103.06

(57) ABSTRACT

The invention is directed to a catheter assembly and method for making the same. The catheter includes an elongated shaft and an inflatable balloon on a portion of a distal shaft section and in surrounding relation thereto. The balloon has proximal and distal tapered regions and an intermediate region longitudinally disposed therebetween. The balloons distal tapered region has a length at least equal to the proximal tapered region length, preferably greater. The proximal and distal tapered regions each forms a taper outer angle with the catheter shaft with the distal taper outer angle being greater than the proximal taper outer angle.

### FIELD OF INVENTION

The invention relates to the field of intravascular delivery systems, and more particularly to balloons for angioplasty.

### BACKGROUND OF THE INVENTION

In percutaneous transluminal coronary angioplasty (PTCA) procedures, a guiding catheter is advanced until the distal tip of the guiding catheter is seated in the ostium of a desired coronary artery. A guide wire, positioned within an inner lumen of an dilatation catheter, is first advanced out of the distal end of the guiding catheter into the patient's coronary artery until the distal end of the guide wire crosses a lesion to be dilated. Then the dilatation catheter having an inflatable balloon on the distal portion thereof is advanced into the patient's coronary anatomy, over the previously introduced guide wire, until the balloon of the dilatation catheter is properly positioned across the lesion. Once properly positioned, the dilatation balloon is inflated with liquid one or more times to a predetermined size at relatively high pressures (e.g. greater than 8 atmospheres) so that the stenosis is compressed against the arterial wall and the wall expanded to open up the passageway. Generally, the inflated diameter of the balloon is approximately the same diameter as the native diameter of the body lumen being dilated so as to complete the dilatation but not overexpand the artery wall. After the balloon is finally deflated, blood flow resumes through the dilated artery and the dilatation catheter can be removed therefrom.

In such angioplasty procedures, there may be restenosis of the artery, i.e. reformation of the arterial blockage, which necessitates either another angioplasty procedure, or some other method of repairing or strengthening the dilated area. To reduce the restenosis rate and to strengthen the dilated area, physicians frequently implant an intravascular prosthesis, generally called a stent, inside the artery at the site of the lesion. Stents may also be used to repair vessels having an intimal flap or dissection or to generally strengthen a weakened section of a vessel. Stents are usually delivered to a desired location within a coronary artery in a contracted condition on a balloon of a catheter which is similar in many respects to a balloon angioplasty catheter, and expanded to a larger diameter by expansion of the balloon. The balloon is deflated to remove the catheter and the stent left in place within the artery at the site of the dilated lesion.

In the design of catheter balloons, balloon characteristics such as strength, flexibility and compliance must be tailored to provide optimal performance for a particular application. An important consideration in the design of the dilatation catheter assemblies is the flexibility of the catheter at the distal end of the balloon while maintaining the strength of the bond between the catheter and the balloon material. This flexibility affects the ability of the catheter for negotiating through the patient's vasculature without causing injury thereto.

Therefore, what has been needed is a dilatation balloon catheter with a flexible distal end and while maintaining the integrity of the bond between the catheter and the balloon, and methods for making the same. The present invention satisfies these and other needs.

### SUMMARY OF THE INVENTION

The invention is directed to a catheter assembly and method for making the same. The catheter includes an elongated shaft having proximal and distal sections, and further includes an inflatable balloon on a portion of the distal shaft section and in surrounding relation thereto. The balloon has proximal and distal tapered regions and an intermediate region longitudinally disposed therebetween. A fluid-tight bond is formed between the catheter shaft and at least a portion of at least one of the proximal and distal tapered regions, preferably, the distal tapered region. The bond is preferably a fusion bond. The balloons of the present invention do not include a distal shaft and as such have greater flexibility at the shaft distal end. The balloons distal tapered region has a length at least equal to the proximal tapered region length, preferably greater. The proximal and distal taper lengths range, from about 1.25 to about 6 millimeter (mm), preferably, from about 2.5 to about 5 mm, and most preferably, from about 3 to about 4.5 mm. In a presently preferred embodiment, the proximal and distal taper lengths range, respectively; from about 1.25 to about 1.75 mm, and from about 5.75 to about 6.25 mm; more preferably, from about 2.25 to about 2.75 mm, and from about 4.75 to about 5.25 mm; and most preferably, from about 2.75 to about 3.25 mm, and from about 4.25 to about 4.75 mm.

The proximal and distal tapered regions form with the catheter shaft, proximal and distal taper angles. The relationship between the proximal and distal taper angles, and the proximal and distal taper lengths, respectively, is that shown in Equation I, below:

Angle°=[(D−0.58)/(2*taper length)][180/π] Equation I

wherein

Angle°=proximal or distal taper angle (degrees°)

D=inflated balloon nominal diameter (mm)

Taper Length=proximal or distal taper length (mm)

For example, in one embodiment, for a balloon having a nominal inflated diameter of about 3 mm, the proximal and distal taper angles, range from about 39.4 to about 11.6°, preferably, from about 22.3 to about 15.3°. In a presently preferred embodiment, the distal taper angle is greater than the proximal taper angle by about 27.8 to about 7°.

In the process of manufacturing the distally shaftless balloons of the present invention with the fluid-tight seal between the balloon distal tapered region and the catheter shaft, the distal shaft of the balloon is removed at the second end of the distal tapered region such that the balloon material at least substantially terminates at the second end of the tapered region. A protective is placed sleeve at the balloon distal tapered region covering, at least in part, the balloon tapered region including the second end thereof and the shaft distal end. Substantially monochromatic energy, at a wave length of maximum spectral absorption of the materials forming the balloon and the distal section of the catheter shaft, is controllably directed onto the distal catheter shaft and at least a section of the distal tapered region at its second end. The concentrated monochromatic energy produces sufficient heat to melt the materials and forms a bond site between the distal catheter shaft and the at least a section of the distal tapered region. As the material is cooled, it solidifies to form a fusion bond between the distal catheter shaft and the balloon. The sleeve is thereafter removed.

In manufacturing the balloons, it is desirable to have balloons with sufficiently similar proximal and distal taper lengths. During the manufacturing process, the directed laser beam can have variations in its focal width (the width of the beam at the desired area) resulting in variations in the length of the bond, thus, the final length of the distal tapered region.

In providing a robust manufacturing process, a balloon with unsealed distal end is provided having a distal tapered region length at least equal to, and preferably, longer, than the proximal tapered region length. As such, the length of the distal tapered region, after sealing, will be at least equal to the proximal tapered region length. This is especially of interest, when the balloons are formed of material which are more susceptible to shrinkage during the sealing process, as for example with polyurethane balloons.

### BRIEF DESCRIPTION OF THE DRAWINGS

FIG. 1 is an elevational view of a balloon catheter embodying features of the invention.

FIG. 2 is a longitudinal cross-sectional, partially cut away, view of the catheter shown in FIG. 1 taken within lines 2-2.

FIG. 3 is a cross sectional view of the balloon catheter of FIG. 2 taken along lines 3-3.

FIG. 4 is a longitudinal cross-sectional, partially cut away, view of an unsealed balloon used to make the balloon of the catheter of FIG. 1.

FIGS. 5A through 5E show a preferred process for forming the catheters of the present invention.

### DETAILED DESCRIPTION OF THE INVENTION

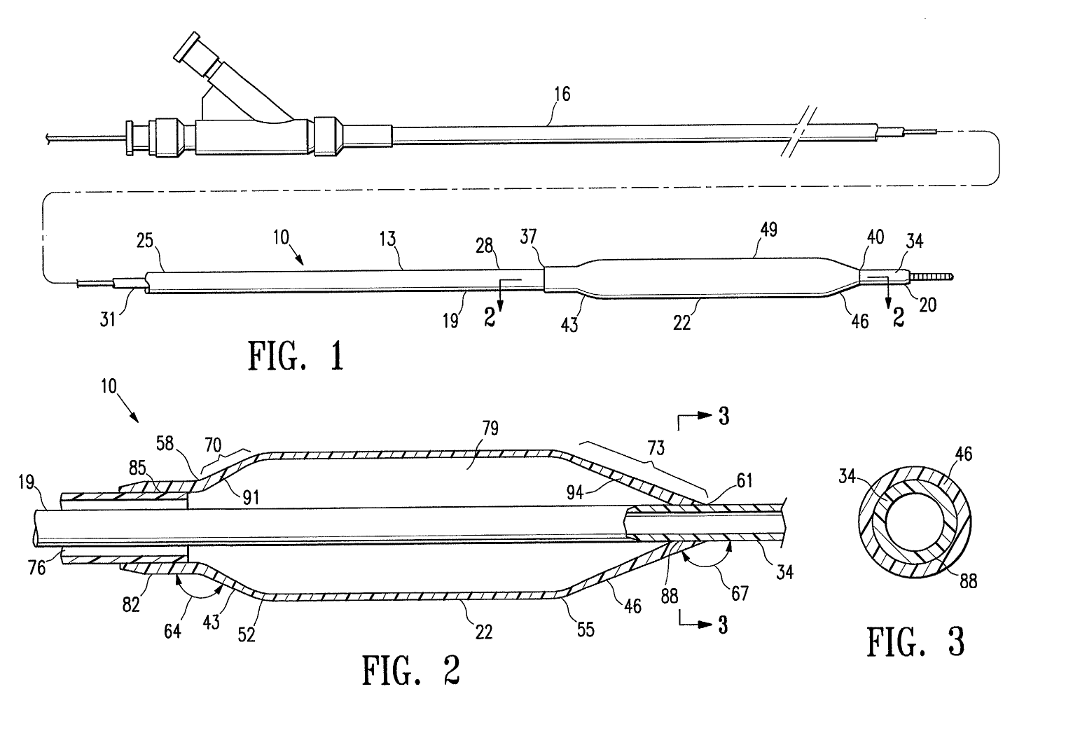

In the embodiment features of which are illustrated in FIG. 1, the balloon catheter 10 of the present invention includes an elongated catheter shaft 13 having a proximal section 16 and a distal section 19 with a distal end 20, and an inflatable balloon 22 on the distal section 19 of the shaft 13 and in surrounding relationship thereto. The catheter shaft 13 comprises an outer tubular member 25 having a distal portion 28; and an inner tubular member 31 having an inner lumen 32 extending therein configured to slidably receive a guidewire 33 suitable for advancement through a patient's coronary arteries, and a distal portion 34. The balloon 22 has proximal and distal ends 37 and 40; proximal and distal tapered regions 43 and 46, and an intermediate region 49 longitudinally disposed between the proximal and distal tapered regions 43 and 46. The proximal and distal tapered regions 43 and 46 each has a first end 52 and 55, respectively, adjacent the intermediate region 49; and a second end 58 and 61, opposite their respective first ends, 52 and 55. The balloon proximal and distal tapered regions, 43 and 46, form with the catheter shaft 13, proximal and distal taper angles, 64 and 67, the distal taper angle 67, preferably, being larger than the proximal taper angle 64. A longitudinal dimension 70 of the balloon proximal tapered region 43 is, preferably, equal to or less than a longitudinal dimension 73 of the balloon distal tapered region 46. A stent (not shown) may be mounted on at least a portion of the intermediate region 49 to form a stent delivery catheter system.

As best illustrated in FIG. 2, an inflation lumen 76 formed between the outer tubular member 25 and the inner tubular member 31 is in fluid communication with a balloon interior 79. Preferably, a balloon proximal shaft 82 extends between the balloon proximal taper second end 58 and the balloon proximal end 37. The balloon 22 is sealingly secured to the shaft 13 by one or more bonds, preferably, fusion bonds 85 and 88, at or near the balloon proximal shaft 82 and the distal portion 28 of the outer tubular member 25, and the balloon distal taper second end 61 and the distal portion 34 of the inner tubular member 31, respectively.

The proximal and distal taper lengths, 70 and 73, range, from about 1.25 to about 6 millimeter (mm), preferably, from about 2.5 to about 5 mm, and most preferably, from about 3 to about 4.5 mm. In a presently preferred embodiment, the proximal and distal taper lengths range, respectively; from about 1.25 to about 1.75 mm, and from about 5.75 to about 6.25 mm; more preferably, from about 2.25 to about 2.75 mm, and from about 4.75 to about 5.25 mm; and most preferably, from about 2.75 to about 3.25 mm, and from about 4.25 to about 4.75 mm.

The proximal and distal taper angles, 64 and 67, preferably, have a relationship with the proximal and distal taper lengths, respectively, as shown in Equation I, below:

Angle°=[(D−0.58)/(2*taper length)][180/π] Equation I

wherein

Angle°=proximal or distal taper angle (degrees°)

D=inflated balloon nominal diameter (mm)

Taper Length=proximal or distal taper length (mm)

For example, in one embodiment, for a balloon having a nominal inflated diameter of about 3 mm, the proximal and distal taper angles, range from about 39.4 to about 11.6°, preferably, from about 22.3 to about 15.3°. In a presently preferred embodiment, the distal taper angle is greater than the proximal taper angle by about 27.8 to about 7°.

The longitudinal dimension of the proximal and distal fusion bonds 85 and 88, independently, can range from about 0.25 to about 10 millimeters (mm), preferably, from about 1 to about 7 mm; depending on the presence and configuration of other components, as described in more detail below. The distal fusion bond 88 has a proximal longitudinal dimension extending along at least a portion of the distal tapered region 46 of the balloon 22 toward the intermediate region 49, and ranges from about 0.05 to about 1 mm; preferably from about 0.2 to about 0.3 mm. The tapered regions 43 and 46 each has a wall thickness 91 and 94 respectively, which may increase from their respective first ends, 52 and 55, to their respective second ends, 58 and 61.

Alternatively, the proximal portion of the balloon can be coextruded with the outer tubular member 25, the balloon forming a distal bond with the distal portion of the inner member.

The balloon 22 may be formed from thermoplastic elastomers (TPE) with various properties.

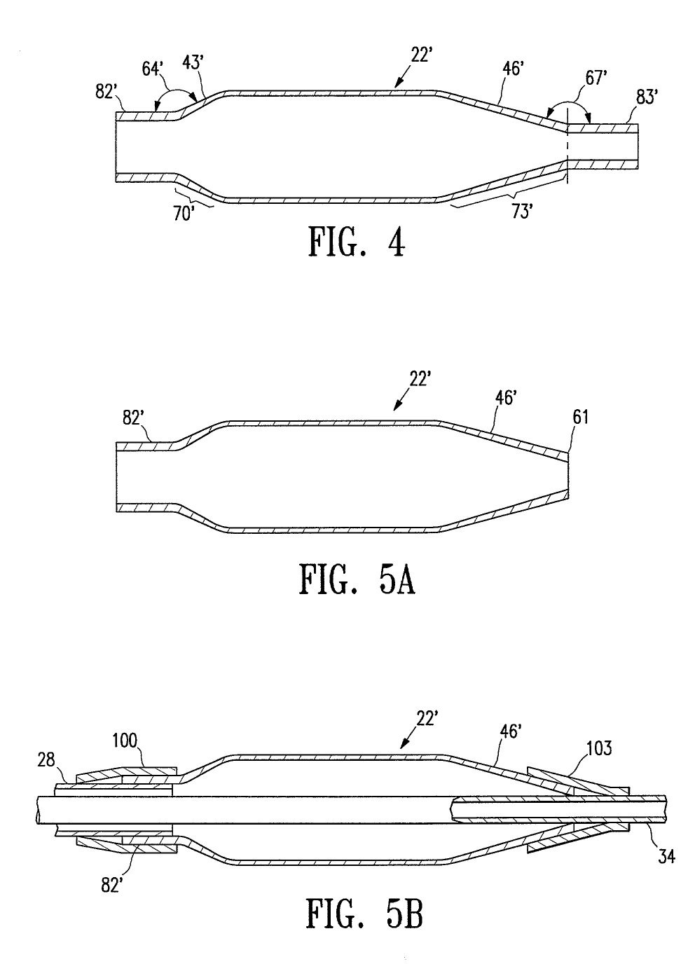

By way of forming the catheter assembly of the present invention, such as catheter assembly 10 in FIG. 1, wherein like references indicate like elements, a balloon 22′, as shown in FIG. 4, is provided, the balloon 22′ having unsealed proximal and distal shafts 82′ and 83′. The unsealed balloon 22′ generally has proximal and distal tapered regions 43′ and 46′ having longitudinal dimensions, 70′ and 73′, at least equal to their respective sealed proximal and distal tapered regions 43 and 46.

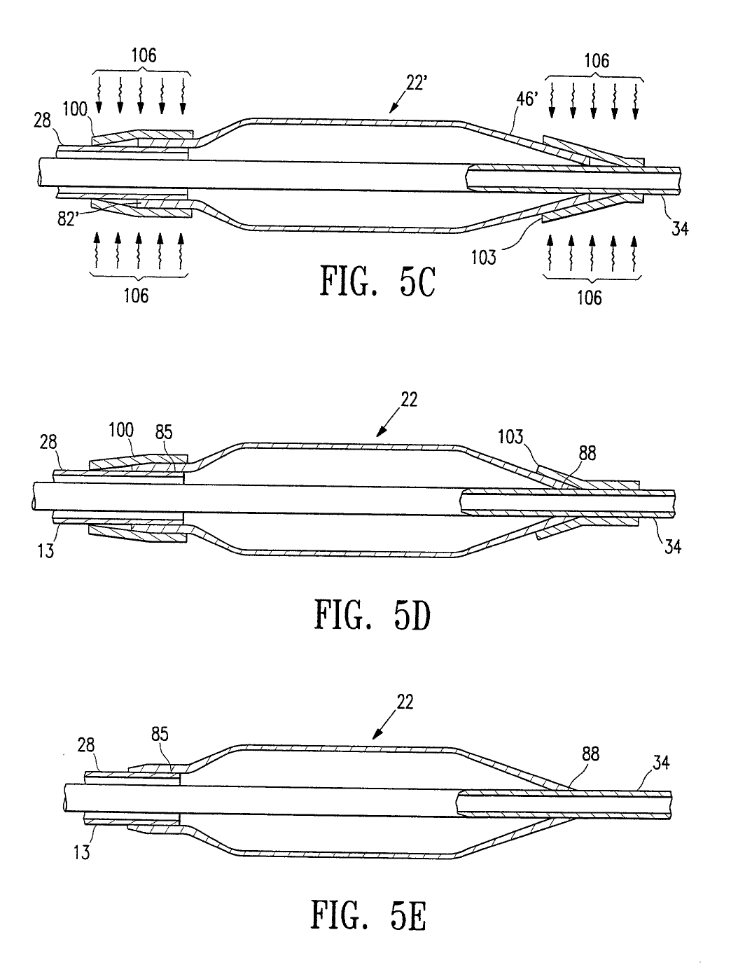

The proximal and distal tapered regions, 43′ and 46′, of the distally unsealed balloon 22′, form, with the catheter shaft 13, proximal and distal taper angles, 64′ and 67′. Now referring to FIGS. 5A through 5E, the distal shaft 83′ is removed at the second end 61 of the distal tapered region 46′ such that the balloon material at least substantially terminates at the distal second end 61.

Shrink tubings 100 and 103 are placed around at least a portion of the unsealed proximal shaft 82′, and the unsealed distal tapered region 46′, respectively.

Substantially monochromatic energy from a heat source 106 at a wavelength of maximum spectral absorption of the materials forming the balloon and the distal section of the catheter shaft is controllably applied to the desired interface to be bonded (e.g., interface between the unsealed proximal shoulder 82′ and the distal outer member 28, and the second end 61 of the balloon and the distal inner member 34) producing sufficient heat to melt the materials at the desired interfaces.

The materials forming the distal catheter shaft and the balloon along the bond site and the immediate region thereof are then melted. The melted area is then cooled forming proximal and distal bonds 85 and 88

The shrink tubings 100 and 103 are then removed, forming the balloon 22 sealed to the shaft 13, according to that described above. The bonded interfaces, preferably, have a crystallinity greater than the crystallinity of the starting unsealed material.

The presently preferred fusion heat source 106 is a CO₂ laser. The laser power is about 50 mW to about 250 mW, the laser rotation speed about the members to be bonded is about 75 to about 300, and the laser absolute focus is about 0.30 to about 0.50. The materials are heated at temperatures between about 100° C. to about 200° C. for about 30 to about 150 seconds.

In one embodiment, the balloon is formed from compliant material, compliant at least within a working range of the balloon, and which therefore provides for substantially uniform radial expansion within the working range. The term “compliant” as used herein refers to thermosetting and thermoplastic polymers which exhibit substantial stretching upon the application of tensile force. The compliant balloon material expands substantially elastically when pressurized at least within the operating pressure range for the present system. Additionally, compliant balloons transmit a greater portion of applied pressure before rupturing than non-compliant balloons. Suitable compliant balloon materials include, but are not limited to, elastomeric materials, such as elastomeric varieties of latex, silicone, polyurethane, polyolefin elastomers, such as polyethylene, flexible polyvinyl chloride (PVC), ethylene vinyl acetate (EVA), ethylene methylacrylate (EMA), ethylene ethylacrylate (EEA), styrene butadiene styrene (SBS), and ethylene propylene diene rubber (EPDM). The presently preferred compliant material has an elongation at failure at room temperature of at least about 250% to at least about 500%, preferably about 300% to about 400%, and a Shore durometer of about 50A to about 75D, preferably about 60A to about 65D.

Alternatively, the balloon can be formed from semi-compliant material, the semi-compliant material formed at least in part of a block copolymer, such as a polyurethane block copolymer. As used herein, the term semi-compliant balloon refers to a balloon with low compliance, exhibiting moderate stretching upon the application of tensile force. The semi-compliant balloon has a compliance of less than about 0.045 millimeters/atmosphere (mm/atm), to about rupture, in contrast to compliant balloons such as polyethylene balloons which typically have a compliance of greater than 0.045 mm/atm. The percent radial expansion of the balloon, i.e., the growth in the balloon outer diameter divided by the nominal balloon outer diameter, at an inflation pressure of about 150 psi (10.2 atm) is less than about 4%.

The presently preferred semi-compliant material is a polyurethane block copolymer. Suitable polyurethane block copolymers include polyester based polyurethanes such as PELLETHANE available from Dow Plastics and ESTANE available from B F Goodrich, polyether based aromatic polyurethanes such as TECOTHANE available from Thermedics, polyether based aliphatic polyurethanes such as TECOPHILIC available from Thermedics, polycarbonate based aliphatic polyurethanes such as CARBOTHANE available from Thermedics, polycarbonate based aromatic polyurethanes such as BIONATE available from PTG, solution grade polyurethane urea such as BIOSPAN available from PTG, and polycarbonate-silicone aromatic polyurethane such as CHRONOFLEX available from Cardiotech. Other suitable block copolymers may be used including TEXIN TPU available from Bayer, TECOPLAST available from Thermedics, and ISOPLAST available from Dow.

The balloons of the invention can also be made of polyamide/polyether block copolymers. The polyamide/polyether block copolymers are commonly identified by the acronym PEBA (polyether block amide). The polyamide and polyether segments of these block copolymers may be linked through amide linkages, however, most preferred are ester linked segmented polymers, i.e. polyamide/polyether polyesters. Such polyamide/polyether/polyester block copolymers are made by a molten state polycondensation reaction of a dicarboxylic polyamide and a polyether diol. The result is a short chain polyester made up of blocks of polyamide and polyether. The polyamide and polyether blocks are not miscible. Thus the materials are characterized by a two phase structure: one is a thermoplastic region that is primarily polyamide and the other is elastomer region that is rich in polyether. The polyamide segments are semicrystalline at room temperature. The generalized chemical formula for these polyester polymers may be represented by the following formula:

OH—(CO—PA—CO—O—PE—O)ₙ—H

in which PA is a polyamide segment, PE is a polyether segment and the repeating number n is between 5 and 10. The polyamide/polyether polyesters are sold commercially under the PEBAX® trademark by companies such as Elf Atochem North America Inc. of Philadelphia, Pa. Examples of suitable commercially available polymers are Pebax® 33 series polymers. The suitable material for the balloon, atraumatic tip, and collar preferably have different hardness values and are selected to provide the necessary flexibility. For example, the balloon material may be selected from material with hardness 60 and above, Shore D scale, more preferably, Pebax® 7033 and 7233.

The inner tubular member of the shaft may be formed of any suitable material compatible with the material to which it will be fusion bonded. For example, the inner tubular member may be a multilayer tubular member with a first or outer layer which is fusion bondable to one or more of the materials for forming the balloon, and a second or inner layer which has lubricious properties. A high strength outer layer may be bonded to at least part of the first layer to provide additional strength and pushability. The first layer should have a melting point which is at least 20° C., preferably at least 30° C. lower than the melting point of an adjacent polymeric layer, so that the adjacent layer is not distorted by the heat from the fusion bonding procedure.

The material from which the first layer of the multilayered member, which has a lower melting point than the adjacent second layer, is selected so as to be compatible with the polymeric material of the catheter component to which it is to be secured (e.g., balloon). A presently preferred lower melting point polymeric material is a polyolefin based copolymer with not more than 35% (by weight) reactive monomer forming the copolymer. A suitable polyolefin material is copolymerized with one or more monomers selected from the group consisting of carboxylic acid or acrylic acid or anhydride thereof and preferably is unsaturated. A presently preferred polyolefinic material is a polyethylene based adhesive polymer such as ethylene-acrylic acid copolymer which is sold commercially as PRIMACOR by Dow Chemical Co. or as ESCOR by EXXON or as PLEXAR by Quantum Chemical Corp. Other suitable materials include polymers which have been modified by reactive extrusion having a durometer range of about Shore A 80 to about Shore D 80, preferably about Shore A 90 to about Shore D 70.

The second or inner layer of the multilayer member having lubricious properties should have a coefficient of friction (both static and dynamic) of less than 0.35 and preferably less than 0.30. Suitable polymeric materials having the aforesaid coefficient of friction include polyethylene, polytetrafluoroethylene and other fluoropolymers.

A third layer may be provided on the side of the first layer opposite side in contact with the second layer and may be formed of various polymeric materials to provide a catheter shaft with additional push and to prevent collapse or kinking of the tubular member in manufacturing or use. Suitable polymeric materials for the third layer include high density polyethylene, polyethylene terephthalate (PET), polyamide, a thermoplastic polyurethane, polyetheretherketone (PEEK) and the like.

All or most of the layers of the multilayered tubular member are preferably selected or modified so that they can be melt processed, e.g. coextruded, simultaneously or sequentially, and as a result the polymeric materials of the various layers should be compatible in this regard or made compatible by appropriate additives to the polymers.

The outer tubular member 25 may be formed of a polymeric material, including nylons; polyether block amides such as Pebax; polyurethanes; polyester block copolymers (containing one or more of the following glycols) comprising hard segments of polyethylene-terephthalate or polybutylene-terephthalate, and soft segments of polyether such as polyethylene glycol, polypropylene glycol or polytetramethylene glycol ethers, such as those available under the trade name Hytrel; polyesters under the trade name Arnitel; or blends thereof. The outer tubular member 67 is preferably formed at least in part of Nylon 12.

While particular forms of the invention have been illustrated and described, it will be apparent that various modifications can be made without departing from the spirit and scope of the invention. Accordingly, it is not intended that the invention be limited, except as by the appended claims.

What is claimed is:

1. A catheter assembly, comprising:
an elongated shaft having proximal and distal sections;
an inflatable balloon on a portion of the distal shaft section and in surrounding relation thereto, the balloon having proximal and distal tapered regions and an intermediate region longitudinally disposed therebetween, the proximal and distal tapered regions each having a first end adjacent the intermediate region and a second end opposite the first, the proximal and distal tapered regions each forming an taper angle with the catheter shaft with the distal taper angle being greater than the proximal taper angle; and
a fluid-tight bond between the catheter shaft and at least a section of at least one of the proximal and distal tapered regions at the second end thereof.

2. The assembly of claim 1 wherein the proximal and distal taper angles have a dimension, independently determined by Equation I:
Angle°=[(D−0.58)/(2*taper length)][180/π] Equation I
wherein
Angle°=proximal or distal taper angle (degrees°)
D=inflated balloon nominal diameter (mm)
Taper Length=longitudinal dimension of the proximal or distal taper region (mm) corresponding to the proximal or distal taper angle, respectively,

3. The assembly of claim 1 wherein the proximal and distal taper angles have a dimension, independently ranging from about 39.4 to about 11.6 degrees.

4. The assembly of claim 1 wherein the proximal and distal taper angles have a dimension, independently ranging from about 22.3 to about 15.3 degrees.

5. The assembly of claim 1 wherein the distal taper angle is greater than the proximal taper angle by about 27.8 to about 7 degrees.

6. A catheter assembly, comprising:
an elongated shaft having proximal and distal sections;
an inflatable balloon on a portion of the distal shaft section and in surrounding relation thereto, the balloon having proximal and distal tapered regions and an intermediate region longitudinally disposed therebetween, the proximal and distal tapered regions each having a first end adjacent the intermediate region and a second end opposite the first end, the distal tapered region having a longitudinal dimension equal to or greater than the same for the proximal tapered region; and
a fluid-tight bond between the catheter shaft and at least a section of at least one of the proximal and distal tapered regions at the second end thereof.

7. The assembly of claim 6 wherein the distal tapered region has a longitudinal dimension greater than the same for the proximal tapered region.

8. The assembly of claim 6 wherein the longitudinal dimension of the proximal and distal tapered lengths, independently, range, from about 1.25 to about 6 millimeter (mm).

9. The assembly of claim 6 wherein the longitudinal dimension of the proximal and distal tapered lengths, independently, range, from about 2.5 to about 5 millimeter.

10. The assembly of claim 6 wherein the longitudinal dimension of the proximal and distal tapered lengths, independently, range, from about 3 to about 4.5 millimeter.

11. The assembly of claim 6 wherein the longitudinal dimension of the proximal and distal tapered lengths range, respectively, from about 1.25 to about 1.75 millimeter, and from about 5.75 to about 6.25 millimeter.

12. The assembly of claim 6 wherein the longitudinal dimension of the proximal and distal tapered lengths range, respectively, from about 2.25 to about 2.75 millimeter, and from about 4.75 to about 5.25 millimeter.

13. The assembly of claim 6 wherein the longitudinal dimension of the proximal and distal tapered lengths range, respectively, from about 2.75 to about 3.25 millimeter, and from about 4.25 to about 4.75 millimeter.

14. A method for forming a catheter assembly, the method comprising:
providing an elongated shaft having proximal and distal sections and a distal tip;
providing an inflatable balloon on a portion of the distal shaft section and in surrounding relation thereto, the balloon having proximal and distal tapered regions and a intermediate region longitudinally disposed therebetween, the proximal and distal tapered regions each having a first end adjacent the intermediate region and a second end opposite the first end, the distal tapered region having a longitudinal dimension greater than the same for the proximal tapered region; the balloon further having proximal and distal shaft regions adjacent the second end of the proximal and distal tapered regions, respectively, and extending away from the corresponding proximal and distal tapered region first ends;
removing the balloon distal shaft region at the second end of the distal tapered region such that the balloon material at least substantially terminates at the second end of the tapered region;
providing a protective sleeve at the balloon distal tapered region, the protective sleeve covering, at least in part, the balloon tapered region including the second end thereof and the shaft distal tip;
providing substantially monochromatic energy at a wave length of maximum spectral absorption of the materials forming the balloon and the distal section of the catheter shaft;
controllably directing the monochromatic energy onto the distal catheter shaft and at least a section of the distal tapered region at the second end thereof to concentrate the monochromatic energy to form a bond site between the distal catheter shaft and the at least a section of the distal tapered region;
melting at least a portion of the materials forming the distal catheter shaft and the balloon along the bond site and the region immediately adjacent thereto; allowing the previously melted materials to cool and solidify to form a fusion bond between the distal catheter shaft and the balloon; and
removing the protective sleeve.

15. The method of claim 14 wherein the balloon providing step further includes the proximal and distal tapered regions each forming a taper outer angle with the catheter shaft with the distal taper outer angle being greater than the proximal taper outer angle.
